(19) United States
(12) Patent Application Publication
(10) Pub. No.: US 2020/0139575 A1
(43) Pub. Date: May 7, 2020

(54) Device For The Thermal Treatment Of Logs

(71) Applicant: THERMODYNAMIC WORKSHOP TRAINING - TWT, Le Havre (FR)

(72) Inventors: Philippe PINAULT, Amanlis (FR); Valentin PINAULT, Amanlis (FR); Anthony-Ange GATT, Le Havre (FR)

(73) Assignee: ThermoDynamic Workshop Training - TWT, Le Havre (FR)

(21) Appl. No.: 16/495,946

(22) PCT Filed: Mar. 20, 2018
(86) PCT No.: PCT/EP2018/056998

§ 371 Date: Sep. 20, 2019

(30) Foreign Application Priority Data
Mar. 20, 2017 (FR) 1770267

(51) Int. Cl. B27K 5/00; A61L 2/07

(52) U.S. Cl. CPC B27K 5/001; F26B 1/00; A61L 2/07

(57) ABSTRACT

This invention concerns a heat treatment device intended to be associated with an intermodal container (3) loaded with logs, comprising a pressurized water steam supply line (1). Said heat treatment device is particular in that it comprises at least one, preferably two, substantially rectilinear tube (2), provided with a plurality of openings distributed over its length and circumference, said at least one tube (2) being intended to be introduced from the rear into said intermodal container (3) loaded with logs, and to be supplied by said pressurized water steam.

This invention also concerns a method that can be implemented by means of a device depending on the invention.

The present invention is in the field of phytosanitary treatment of logs, in particular hardwood and softwood logs. It concerns more particularly a device for heat treatment under the bark of logs.

Log transport is involved in the spread of plant pests, including fungi and insects. It is therefore necessary to treat the logs in order to limit this phenomenon. Such phytosanitary treatment is even mandatory for the export of logs to certain countries. According to current standards, this treatment consists in keeping all logs under bark (under cortical) at a temperature above 71.1° C. for at least 75 minutes. A heat treatment, consisting of the injection into a log intermodal container of hot air or steam streams at a temperature between 80 and 150° C., is usually carried out. The injection is done from the back of the intermodal container, after opening the doors. As the heat comes from the back of the intermodal container, this method does not allow a homogeneous distribution of heat in the intermodal container, and results in high energy consumption. The heat treatment cycle is also long, due to a long heating time under the bark of the logs furthest from the heat source.

U.S. Pat. No. 5,447,686 discloses a method for the heat treatment of wood pieces with steam to remove various parasites. The wooden pieces are loaded into the hold of a boat equipped for steam production. The pieces are brought into contact with the steam, injected from the bottom of the hold, to raise the temperature and maintain it at a sufficient level for a sufficient time to remove any parasites that may be present. However, to apply this method, the wood cannot remain in an intermodal container but must be unloaded into the ship's hold.

The purpose of this invention is to compensate at least in part for these disadvantages by proposing a device for treating logs in an intermodal container without removing the logs from said intermodal container. The operation can take place in the intermodal container still on the semi-trailer attached to its tractor, without any handling of the logs. The device according to the invention also allows a rapid heating of the logs at any location in the intermodal container.

To this end, the present invention proposes a heat treatment device to be associated with an intermodal container loaded with logs, comprising a pressurized water steam inlet line. Said heat treatment device is particular in that it comprises at least one, preferably two, substantially straight tube, provided with a plurality of openings distributed over its length and circumference, said at least one tube being intended to be introduced from the rear into said intermodal container loaded with logs, and to be supplied by said pressurized water steam.

Thanks to these provisions, the heat injection being distributed over the entire length of the intermodal container, the heat treatment of the logs is fast and optimal in terms of energy consumption. The device also allows an easy heat treatment to be implemented in a standard intermodal container as used for transport by truck or ship.

According to other features:
said openings of said tubes are of increasing size and/or number from the water steam supply side, improving the distribution of steam in the intermodal container so that logs located at the end of the intermodal container opposite to the steam supply of the tubes, and thus conveying cooler or already condensed steam, can rise in temperature as quickly as the logs located near the supply,
said at least one tube may have an external diameter of less than 25 mm, which allows it to be easily inserted into the intermodal container in the free spaces between the logs,
said device may also comprise a condensed water recovery gutter, allowing the evacuation and recovery of the water produced during the heat treatment by the condensation of the steam, for another use,
said device may also comprise a drain line for evacuating the water recovered in the gutter, said line being provided with a filter, making it possible to recover the water from the gutter for another use, for example the production of water steam to supply the line,
said device may also comprise a sealing device, consisting in particular of at least one foam strip, intended to be placed at the rear opening of said intermodal container in order to reduce the escape of water steam and heat, making it possible to reduce the energy consumption of the device according to the invention; in particular, the left door may be closed again after installation of the device, and said foam strip disposed at the opening of the right door,
said foam strip may be provided with a magnetic strip for fixing it to the intermodal container, which constitutes a simple and easy embodiment of the invention, even if it has to be repeated.

The present invention also concerns a method for the thermal treatment of logs loaded in an intermodal container, characterized in that it comprises the following steps:
opening of the intermodal container from the back,
introduction of at least one tube, so that it extends substantially over the entire length of the intermodal container, in particular over at least three-quarters of said length,
connection of said tube to a water steam supply line,
pressurization of the line.

Before the line is pressurized, the left door of the intermodal container may be closed and the right door can be blocked ajar.

Thanks to these provisions, the heat injection being distributed over the entire length of the intermodal container, the heat treatment of the logs is fast and optimal in terms of energy consumption. Such a heat treatment method is easy to implement in a standard intermodal container as used for transport by truck or ship.

According to other features:
said method may also comprise a step of sealing the door openings with a sealing device before the line is pressurized, making it possible to reduce the energy consumption of the method according to the invention.

This invention will be better understood when reading the following detailed description, with reference to the attached figures in which:

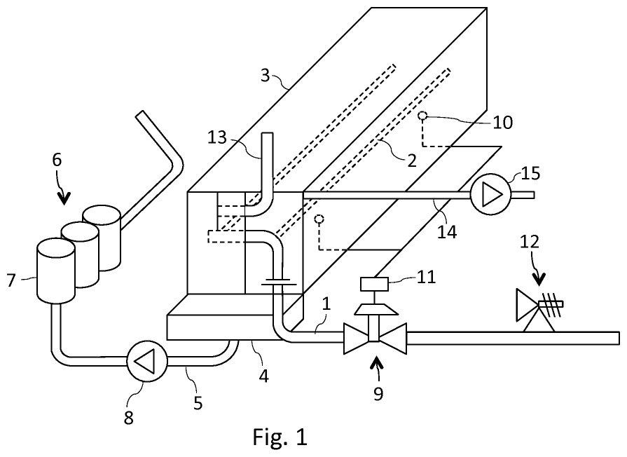

FIG. 1 is a schematic view of the heat treatment device according to the invention.

The heat treatment device according to the invention, illustrated in FIG. 1, comprises a pressurized water steam supply line 1, feeding a tube 2 intended to be introduced into a log intermodal container 3 from the rear.

This device is intended to be introduced into an intermodal container 3, it is therefore separable from said intermodal container 3, and may be used to treat several separate intermodal containers 3 one after the other.

The tube 2 is substantially straight, and has a plurality of openings distributed over its length and circumference. These openings may consist of sawn notches in the tube 2. The tube 2 has a length that can be, for example, about 10 metres, so that once introduced into the intermodal container 3, the water steam escaping through its openings can quickly reach all the logs in the intermodal container 3 and over their entire length. The heat treatment can thus be homogeneous throughout the whole of the intermodal container 3. In order to obtain an optimal result, the tube 2 may be inserted halfway up into the intermodal container 3.

In the preferred embodiment of the invention shown in FIG. 1, the heat treatment device according to the invention comprises two tubes 2, in order to further improve the distribution of water steam in the intermodal container 3. For example, both tubes 2 may be placed halfway up the intermodal container 3 and distributed over its width.

The openings of the tube 2 may be of increasing size and/or number from the end supplied by line 1. This ensures that sufficient water steam reaches the end of the tube 2 furthest from the water steam supply to achieve a uniform thermal environment within the intermodal container 3.

The tube 2 may have an outside diameter of less than 25 mm, so that it can be easily inserted among the logs in the intermodal container 3. However, this outer diameter must be large enough to transport the desired amount of water steam. The thickness of the tube 2 may be at least 2 mm, so that the tube 2 is sufficiently strong and does not deform too much when inserted between the logs of the intermodal container 3, while resisting the pressure exerted within by the water steam. The tube 2 may have a certain elasticity, allowing it, for example, to bypass a log to find its way to the front of the intermodal container. Such elasticity will cause the tube to be slightly deformed, but in the context of this invention it must nevertheless be considered as “substantially straight”.

Partial condensation of steam occurs in tube 2, releasing most of the latent heat without damaging the logs. The rest of the steam condenses on contact with the bark of the logs, ensuring an optimal temperature rise of the logs.

The device according to the invention may further comprise a sealing device, consisting, for example, of one or more foam strips. The sealing device may comprise magnetic strips for fixing it to the intermodal container, and it may be arranged at the rear opening of the intermodal container, in particular at the opening of the right door through which the end of the line 1 and tube 2 are inserted. This allows to reduce the escape of water steam and heat from the intermodal container 3 during heat treatment, and thus reduces the energy consumption of the heat treatment device.

In the embodiment of FIG. 1, the heat treatment device includes a water recovery gutter 4, in the form of a tank placed at the back of the intermodal container 3. The intermodal container 3 can then be tilted, for example by an angle of about 5 to 10 degrees, so that the condensed water and any tannins produced during the heat treatment flow to the gutter 4.

The water collected in the gutter 4 can then be directed to a drain line 5 which may comprise a filter 6. Filter 6 may comprise one or more filter tanks 7, each tank comprising one or more layers of filter material such as filter foam, activated carbon and fine filter wadding. The evacuated water can be sent, for example by means of a diaphragm pump 8, from the gutter 4 to the filter 6 and successively through the different layers of the tanks 7. The filtered water can then be recovered for any purpose, for example, to the boiler producing the water steam of line 1. This boiler may be a waste burning boiler, thus allowing a very low consumption of natural resources.

A hose 14 may be placed at the right door in the corner of the upper hinge of the intermodal container to vacuum the intermodal container enclosure. This allows the cold air to be extracted at the beginning of the treatment and the creation of an on-demand circulation.

Such a vacuum (under slight atmospheric pressure) may be provided by a vacuum pump or an air steam ejector 15 and can be operated by a valve on request.

To reduce the temperature rise delay, and/or to reduce steam consumption, insulation, such as glass wool, may be placed inside on the intermodal container walls, and/or a cover such as a steam barrier film, such as those used as roof insulation, may be placed on top of the logs.

A tube 2 may also be added on top of the logs, if necessary under said cover, to optimize access of the steam to the logs.

The heat treatment method according to the invention is a treatment of logs directly in the intermodal container in which they are loaded. It comprises the following steps:
opening of the intermodal container 3 from the rear, for example one of the doors of the intermodal container 3 may be ajar,
introduction of at least one tube 2, so that it extends substantially over the entire length of the intermodal container 3; for example, if the intermodal container is 12 m long, a 10 m tube may be used,
connection of said tube 2 to a water steam supply line 1,
pressurization of line 1, for example a pressure of at least 5 bar.

An intermodal container 3 sealing step may be added to the previous method just before the line 1 is pressurized, so that the pressurized steam from line 1 that is released into the intermodal container 3 through the tube 2 does not escape largely through the opening of the intermodal container 3.

For example, one of the two rear doors is kept closed, the other is ajar to allow the water steam line 1 and the tubes 2 to pass through. The opening of this door creates an opening space, about 20 mm wide and the height of the door. This space can then be filled with a strip of polymer foam resistant to the temperature and pressure conditions in the intermodal container. Depending on the arrangement of the door in relation to the ceiling and floor of the intermodal container, such a foam strip can also be placed on the top and/or bottom of the door when it is ajar.

A magnetic strip may be placed on the foam strip, allowing the foam strip to be fixed to the intermodal container and easily removed after the treatment, so that it can be reused for another treatment.

The water steam supply line 1 may have a pressure regulator 9. The latter regulates the steam supply in the intermodal container 3 during the heat treatment. The device may then include some temperature sensors 10, placed at certain locations in the intermodal container 3, and a computer 11, in order to automate the control of the pressure regulator 9 and to adjust the steam supply in the intermodal container 3 to the temperature.

Upstream from the pressure regulator 9, the line 1 may have a safety valve 12. In the event of overpressure in the line 1 upstream of the pressure regulator, the valve 12 can lower the pressure to avoid endangering personnel in the vicinity. The valve 12 may be set to 10 bar, for example.

The device may further comprise a «mast riser» type safety device 13 controlling the pressure within the intermodal container 3 to prevent overpressure during heat treatment. The safety device 13 may also simply consist of an interruption of the foam strip to allow the excess steam to escape, this interruption being properly sized to maintain the desired pressure in the intermodal container, without allowing the pressure to rise above acceptable levels.

According to another embodiment, at least one temperature sensor is arranged in the intermodal container enclosure, at a point carefully chosen to give an indication of the lowest temperature in the logs, which makes it possible to monitor that under bark all the locations of the logs are indeed above the prescribed temperature. The pressure regulator can be controlled automatically, but also manually, by increasing the pressure to raise the temperature and decreasing it to lower the temperature, thanks to a simple display of the measured temperature.

Although the above description is based on particular embodiments, it is by no means restrictive of the scope of the invention, and modifications may be made, in particular by substitution of technical equivalents or by a combination different from all or part of the features developed above.

1. A heat treatment device intended to be associated with an intermodal container (3) loaded with logs, the device comprising:
a pressurized water steam supply line (1), having at least one, preferably two, tube (2), provided with a plurality of openings distributed over its length and circumference, said at least one tube (2) being intended to be introduced from the rear into said intermodal container (3) loaded with logs, and to be supplied by said pressurized water steam, wherein said at least one tube (2) being substantially rectilinear, in the sense that said at least tube (2) may have a certain elasticity that will cause the tube (2) to be slightly deformed.

2. The device according to claim 1, wherein said openings of said tubes (2) are of increasing size and/or number starting from the water steam supply side.

3. The device according to claim 1, in which wherein the at least one tube (2) has an external diameter of less than 25 mm.

4. The device according to claim 1, further comprising a gutter (4) for recovering condensed water.

5. The device according to claim 4, further comprising a line (5) for evacuating the water recovered in the gutter (4), said line (5) being provided with a filter (6).

6. The device according to claim 1, further comprising a sealing device, consisting in particular of at least one foam strip, intended to be arranged at the rear opening of said intermodal container (3) in order to reduce the escape of water steam and heat.

7. The device according to claim 6, wherein said foam strip is provided with a magnetic strip for fixing it to the intermodal container (3).

8. A method for the thermal treatment of logs loaded in an intermodal container (3), the method comprising:
opening of the intermodal container (3) from the rear;
introduction of at least one tube (2), provided with a plurality of openings distributed over its length and circumference, so that it extends substantially over the entire length of the intermodal container, in particular over at least three quarters of its length, wherein said at least one tube (2) being substantially rectilinear, in the sense that said at least tube (2) may have a certain elasticity that will cause the tube (2) to be slightly deformed;
connection of said tube (2) to a water steam supply line (1); and
pressurization of the line (1).

9. The method according to claim 8, further comprising a step of sealing the door openings by a sealing device before pressurizing the line (1).
